(19) United States
(12) Patent Application Publication
(10) Pub. No.: US 2021/0310713 A1
(43) Pub. Date: Oct. 7, 2021

(54) ICE MACHINE CLEANING APPARATUS

(71) Applicant: VenMill Industries, Inc., Oxford, MA (US)

(72) Inventors: Christopher Lawrence Bishop, Woodstock, CT (US); Daniel Peter St. Laurent, Webster, MA (US)

(73) Assignee: VenMill Industries, Inc., Oxford, MA (US)

(21) Appl. No.: 17/159,702

(22) Filed: Jan. 27, 2021

Related U.S. Application Data
(60) Provisional application No. 63/004,925, filed on Apr. 3, 2020.

(51) Int. Cl. F25C 1/06; F25C 1/25; C02F 1/68; C02F 1/00; A61L 2/18; A61L 2/26

(52) U.S. Cl. CPC F25C 1/06; A61L 2202/14; C02F 1/68; C02F 1/008; A61L 2/183; A61L 2/26; F25C 2600/02; F25C 2400/12; F25C 2700/12; C02F 2201/782; C02F 2301/046; C02F 2307/12; C02F 2209/40; C02F 2209/23; C02F 2303/04; A61L 2202/15; A61L 2202/23; F25C 1/25

(57) ABSTRACT

Disclosed is a system and method for cleaning an ice machine, a cleaning apparatus comprising an ozone generator, at least one fluid line connecting an output from the ozone generator to at least one of a water inlet, a water recirculatory line, and a water reservoir, wherein the cleaning apparatus is configured for use with an ice machine. In some embodiments, one or more sensors are provided and may be configured to detect a call for new ice formation. The one or more sensors may comprise (i) a flow valve sensor, (ii) a sensor configured to detect if water is flowing through a water inlet, (iii) a sensor configured to detect a beginning of new ice formation, and/or (iv) a sensor configured to detect an end of new ice formation.

### TECHNICAL FIELD

The present disclosure relates generally to cleaning systems for ice machines. More specifically, the present disclosure relates to an ice machine cleaning system that includes an ozone generator.

### BACKGROUND

Ice machines typically include a water pump for pumping fresh water into the system. The water then travels to an evaporator unit comprising a heat exchanger. A compressor pushes refrigerant through the heat exchange pipes of the heat exchanger which will both heat and cool the evaporator when required.

When the ice machine is turned on, the compressor increases the pressure of the refrigerant which raises the temperature. As it passes through the narrow tubes, the refrigerant loses heat to the ambient environment. As the fluid travels through an expansion valve it begins to expand and cool. When this happens, the refrigerant draws heat from the pipes and the evaporator (ice mold). At this point, the water which is flowing over the evaporator begins to freeze.

After the ice cubes form, the evaporator sensor triggers a valve that tells the compressor to stop forcing heated gas into the condenser and instead directs it to a bypass valve. From the bypass valve the hot gas cycles through the evaporator without cooling off and quickly heats up and loosens the ice from the tray without melting it. The ice then falls into the ice bin where it can be scooped by hand or dispensed automatically. Once the ice is dropped, the process starts all over again.

Commercial ice makers are designed to make large quantities of ice and are typically configured to freeze ice from the inside out so that they make clear, uniformly shaped ice.

Ozone (O₃) can be created from oxygen (O₂) in an ozone generator for commercial or industrial applications, however ozone (O₃) quickly reverts back to molecular oxygen (O₂).

### BRIEF DESCRIPTION OF THE DRAWINGS

FIG. 1 illustrates a front perspective view of an ice machine cleaning apparatus, in accordance with an embodiment of the present disclosure.

FIG. 2 illustrates a front perspective view of an ice machine cleaning apparatus highlighting steps in a method for cleaning the ice machine, in accordance with an embodiment of the present disclosure.

FIG. 3 illustrates a method of disinfecting an ice machine, in accordance with some embodiments of the present disclosure.

FIG. 4 illustrates a flow chart illustrating input to one or more sensors, in accordance with some embodiments of the disclosure.

FIG. 5 illustrates a front view of an assembly for disinfecting an ice machine, in accordance with an embodiment of the present disclosure.

FIG. 6 illustrates a front perspective view of an interior portion of an ice machine cleaning apparatus, in accordance with an embodiment of the present disclosure.

FIG. 7 illustrates an interior side view of a portion of an ice machine, in accordance with an embodiment of the present disclosure.

FIG. 8 illustrates an interior side view of a portion of an ice machine, in accordance with an embodiment of the present disclosure.

The figures depict various embodiments of the present disclosure for purposes of illustration only. Numerous variations, configurations, and other embodiments will be apparent from the following detailed discussion.

### DETAILED DESCRIPTION

Disclosed herein is an ice machine cleaning system that includes an ozone generator and control circuitry configured to operate with an ice machine. In some embodiments, the ice machine cleaning system may be integrated into the recirculatory water lines of a refrigerator/freezer. When installed into an ice machine, the system injects ozone into the ice machine's water reservoir to neutralize organics, reduce or eliminate biofilm growth, and disinfect the internal chambers of the ice machine. In some embodiments, the ozone generator and pump provide ozone to at least one of the incoming water supply or directly into the water reservoir, or both. Ambient air is pulled into the system using the pump and ozone may be formed by at least one of corona discharge or UV light. A high voltage corona discharge mechanism or ultra-violet light, for example, can be configured to produce ozone by adding energy to oxygen molecules which causes the oxygen atoms to divide and temporarily recombine with other oxygen molecules, forming ozone. Ozone cannot be stored due to a short half-life and must be produced on-site and on-demand.

Ozonated air is supplied from the ozone generator directly into the ice machine water reservoir and may be recirculated via the water recirculation system of the ice machine. Ozone is bubbled through the water and ozonated water enters into the ice chamber during ice formation. Providing ozone directly into the water supply yields a residence time of the ozone in the water. By diffusing the ozone in the water we have a longer residence time than with just passing ozonated air over the evaporator. The residence time may be less than 20 seconds, less than 40 seconds, less than one minute, or less than two minutes. New ice forms layer by layer as water flows through the evaporator. Thus, freshly ozonated water can be consistently applied to the evaporator during ice formation. Additionally, ozonated water within the ice machine provides cleaning and disinfection to all surfaces that the ozonated water contacts.

Providing ozonated water yields a number of advantages over providing ozonated air. Ozone present in air does not significantly penetrate the water supply. The present disclosure provides a system and method for injecting or infusing ozone directly into the water supply in order to provide a disinfection effect to the water supply and/or to the surfaces that the water supply comes into contact with.

Some of the dissolved or suspended ozone in the water will bubble out of the water and mix with the ambient air within the ice maker. Thus, the system and method described herein provide a disinfection effect both for the water supply directly as well as, in some cases, for the air within the ice machine.

The ice machine cleaning system can stay online and be a permanent part of the ice making machine. In some embodiments, the ozone generation system may be a modular unit configured for use with an ice machine.

In some embodiments, the ozone generator is a corona discharge ozone generator. In some embodiments, the amount of ozone present within the ambient air within the ice maker is greater than 100 μg/m³, greater than 200 μg/m³, greater than 300 μg/m³, between 300 and 500 μg/m³, between 325 and 475 μg/m³, or between 350 and 450 μg/m³ to deter microbial growth. For example, Machery-Nagel Ozone Test Strips may be used for testing ozone levels in ambient air. In some embodiments, the amount of ozone present with the water supply within the ice maker is greater than 0.01 ppm/ltr, greater than 0.025 ppm/ltr, greater than 0.04 ppm/ltr, between 0.01 and 0.6 ppm/ltr, between 0.025 and 0.3 ppm/ltr, or between 0.04 and 0.1 ppm/ltr to deter microbial growth. For example, CHEMets Kits such as Kit K-7404 may be used for testing ozone levels in water.

In some embodiments, the water supply is optionally exposed to ultraviolet (UV) light. For example, incoming water is treated first with ozone and second with ultraviolet (UV) light. In another example, incoming water is treated first with ultraviolet (UV) light and second with ozone. The UV treatment serves to provide secondary disinfection as well as to disinfect water that has been stationary in the system and is low or void of ozone.

### Overview

During the course of operation, ice machines may be susceptible to microbial contamination from slime, mold, bacteria, biofilm, and/or yeast. Many organic compounds are present in the air and may be introduced into an ice machine as the operation of the device pulls in ambient air. Once these compounds come into contact with the surface of an ice machine, the contaminant may proliferate and cause decreased efficiency, total loss of performance, contamination, and/or taste and odor problems.

The water reservoir in an ice machine provides a moisture-rich environment that is a perfect breeding ground for mold, bacteria, and other germs that can greatly affect the quality of the ice being formed in the ice machine. In particular, the water reservoir in the ice maker unit can be susceptible to bacterial growth and contamination. Particularly in a restaurant environment or bakery, yeast is present in the air which can infiltrate the water reservoir and grow into a biofilm. As water is typically pulled directly from the water reservoir to form new ice, this can result in the formation of compromised ice. Compromised ice formed from contaminated water can have a foul taste and/or odor. One sign that the ice machine has been compromised is the presence of an unpleasant odor (e.g., a “musty” smell) coming from the ice machine during operation. Other indicators that an ice machine is compromised include evidence of biofilm or mold growth.

To address the problem of built-up contaminants in ice machines, some users attempt to clean the unit by wiping down the outside of the ice machine's housing or spraying a cleaner onto the unit's components. In other approaches, ozone-rich air is delivered to the ice compartment to reduce mold growth in the ice bin. However, such an approach does not address the ice production part of the ice machine where biofilm growth is even more of a problem—it is very difficult to access and clean inside areas of the ice machine where contaminants reside and grow. In a restaurant setting, for example, the restaurant may rely on a near-continuous supply of ice. Emptying the ice bin and shutting down ice production can disrupt business operations. Accordingly, there is little opportunity to properly clean the ice bin and most ice production components are rarely cleaned, if ever.

Additionally, a need exists for an ice machine cleaning system configured with feedback regarding new ice formation. If ozone is produced and enters a water supply, it will dissipate after a period of time. For example, half of the ozone may dissipate in about 20 seconds, in about 40 seconds, in about one minute, or in about two minutes. To address this problem and others, the present disclosure relates to an ice machine cleaning apparatus configured to provide ozone directly into the water supply and/or water reservoir of the ice machine in order to provide disinfection of the unit. The present disclosure provides a system and method for producing new ozone and supplying it to the water reservoir or water lines at particular times that maximize the disinfection power of the ozone infiltrated in the water. Numerous variations and embodiments will be apparent in light of the present disclosure.

### EXAMPLE EMBODIMENTS

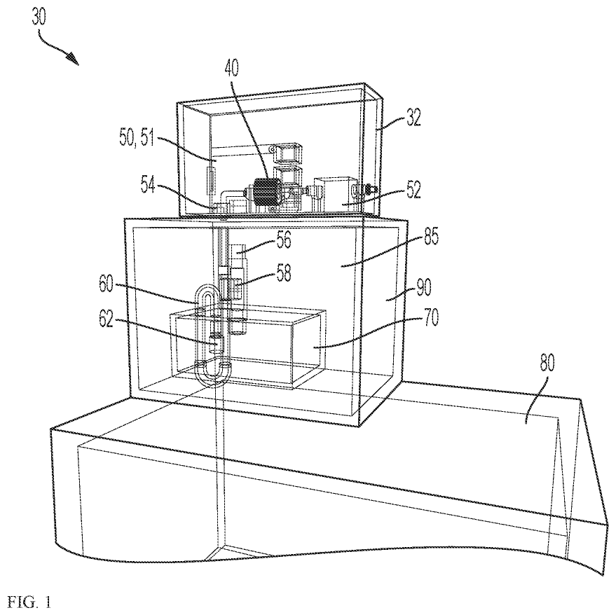

FIG. 1 illustrates a front perspective view of an ice machine cleaning apparatus 30 installed in an ice maker 85, in accordance with an embodiment of the present disclosure. The ice machine cleaning apparatus 30 includes an ozone generator 40, a controller 51, and fittings and other components for integration into the ice maker 85.

The ice machine cleaning apparatus 30 includes ozone generator housing 32. Ozone generator housing 32 contains ozone generator 40 that is configured to pull in ambient air via air pump 52 and generate ozone therefrom. In this example, ozone generator housing 32 is positioned above ice maker housing 90, which is positioned above ice bin 80. Circuit board 50 is configured to control the generation of ozone. In some embodiments, circuit board 50 is in electrical communication with controller 51. Connection port 54 is configured to provide fluid communication between ozone generator housing 32 and ice making housing 90. Connection port 54, in the illustrated embodiment, is positioned between a bottom portion of ozone generator housing 32 and an upper portion of ice maker housing 90.

Ice maker housing 90 comprises ice maker 85 and ozone infusion tube 60, which is connected to silica diffuser 62. In some embodiments, silica diffuser 62 is positioned at the bottom of water reservoir 70 in order to encourage microbubbles of ozone to flow up through the water contained within water reservoir 70. Water flows into ice maker 85 via water inlet 55 (not shown), enters water reservoir 70, and travels to inline water module 56. Flow valve sensor switch 58 is connected to inline water module 56 and is configured to detect changes in water flow during the ice formation cycle. In the illustrated embodiment, the ozone infusion tube is rigid. In some embodiments, ozone infusion tube 60 is a flexible tube, such as a ¼″ flexible hose. In some embodiments, ozone infusion tube 60 is formed from a material that is resistant or substantially resistant to ozone. In most cases ozone infusion tube 60 comprises an ozone-resistant material, such as a urethane laminate, but other materials can be used as deemed suitable for a given application. Ozone infusion tube 60 can be configured in various orientations within ice maker housing 90 and is in fluid communication with inline water module 56 to direct ozone to flow into the water reservoir of the ice maker 85. It is also appreciated that ozone infused into the water may exit the water as a gas and provide ozone-rich air in the ice maker housing 90, thereby disinfecting the air within the ice maker. In some embodiments, a vent is provided to prevent pressure build up. In some embodiments, when flow valve sensor switch 58 detects water flow through inline water module 56 toward water reservoir 70, new ozone is produced and injected to the incoming water supply and/or directly into water reservoir 70. In some embodiments, when flow valve sensor switch 58 detects no water flow through inline water module 56, ozone production halts.

During operation, new ice formation may occur within different time periods depending on the specific construction of the ice maker. In some embodiments, an ice formation cycle may last between about 20 and 45 minutes. During operation, circuit board 50 is configured to call for new ozone production at certain points in the ice making cycle. Circuit board 50 is configured to control operation of ozone generator 40.

In some embodiments, circuit board 50 calls for ozone production when a voltage change is detected by flow valve sensor switch 58. In some embodiments, circuit board 50 is configured to call for ozone production when sensor 59 (not shown) is triggered. Sensor 59 may be at least one of an ozone sensor, a pressure sensor, a flow sensor, and a humidity sensor. In some embodiments, sensor 59 is provided and is configured to detect if water is flowing through water inlet 55. In some embodiments, sensor 59 is configured to detect a change in the ice cycle (i.e. the beginning of new ice formation or the end of new ice formation). In some embodiments, a refresh switch may be provided for a timed production of ozone during lower usage periods. For example, a refresh switch may be desirable during periods of infrequent ice formation or during a period of seasonal disuse. In some embodiments, the refresh switch is a user override button configured external to the ice maker. In some embodiments, a refresh mode may be enabled wherein ozone is periodically generated by the ozone generator. In various embodiments, electrical connection is provided between sensor switch 58, 59, air pump 52, ozone generator 40, and circuit board 50.

The present disclosure provides a system and method for providing water containing at least 0.01 ppm/ltr of ozone in water at all times during operation. In one embodiment, when the ozone level drops below 0.01 ppm/ltr in water or 90 μg/m³ in air, the recirculation system is activated to maintain ozone at a level of greater than 0.01 ppm/ltr in water or 90 μg/m³ in air. The present disclosure provides a system and method for providing ice cubes which are formed using water that has been treated with ozone, resulting in clean water. The present disclosure provides a system and method for providing an improved taste to the ice cubes and a disinfection effect to the ice machine cleaning apparatus.

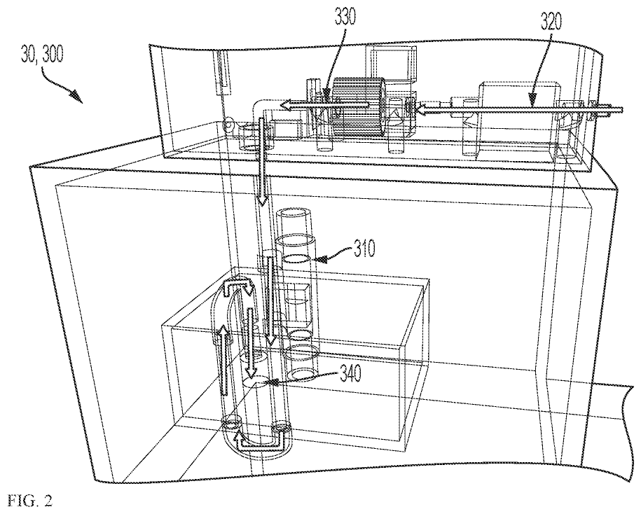

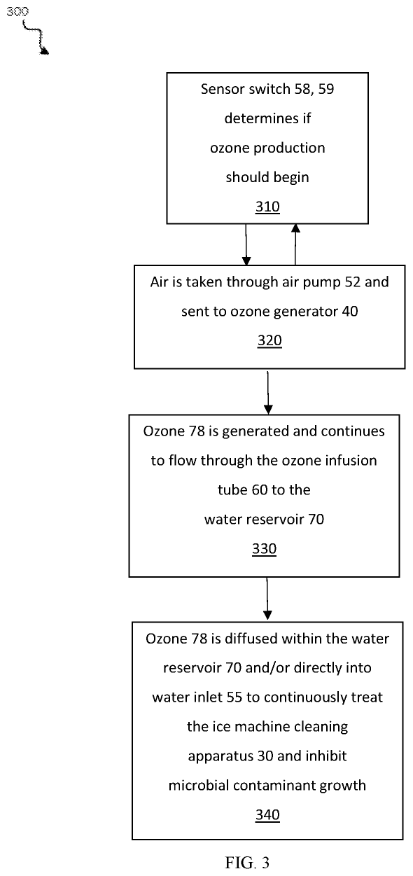

Referring now to FIG. 2, a front perspective view of ice machine cleaning apparatus 30 shows an exploded view of FIG. 1. In FIG. 2, the location of the steps of method 300 are illustrated where they occur within ice machine cleaning apparatus 30. FIG. 3 illustrates a flow chart of the method 300 of disinfecting an ice machine cleaning apparatus 30, in accordance with some embodiments of the present disclosure.

Step 310 occurs between one or more sensors 58, 59 and circuit board 50 to control the production of ozone. In step 310, which comprises a feedback loop, sensor switch 58, 59 checks if ice maker 85 begins running an ice making cycle. In step 320, air is flowed between air pump 52 and ozone generator 40. In step 330, ozone 78 is generated and continues to flow through ozone infusion tube 60 to the water reservoir 70. In step 340, ozone 78 is diffused within the water reservoir 70 and/or directly into water inlet 55 to continuously treat the ice maker system and inhibit microbial contaminant growth.

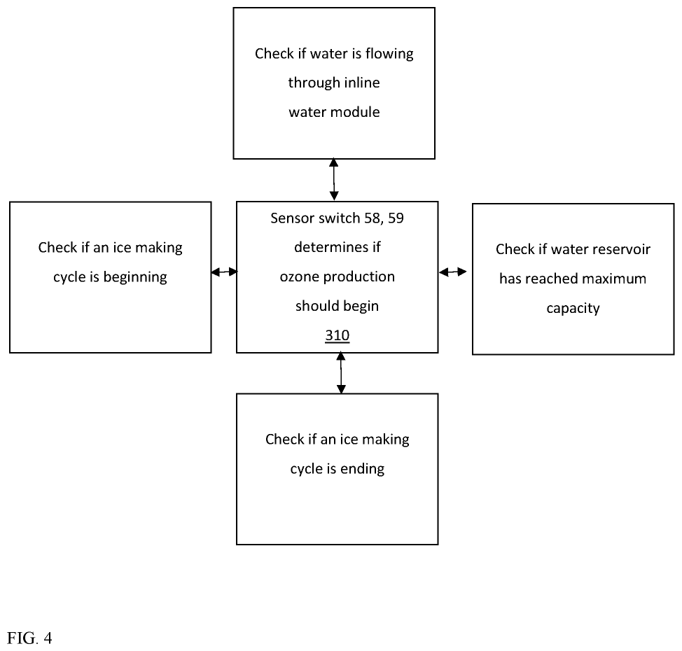

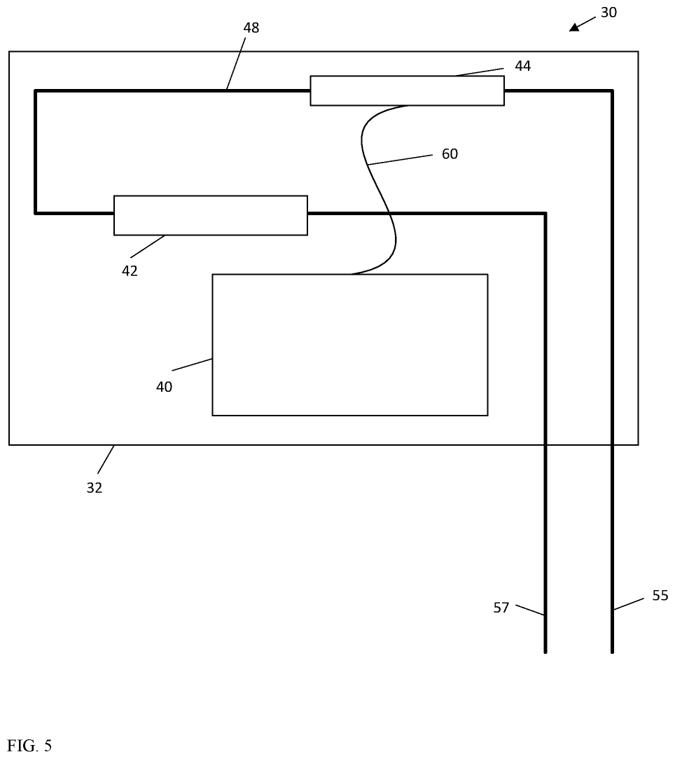

FIG. 4 illustrates a flow chart illustrating input to one or more sensors, in accordance with some embodiments of the disclosure. Input is received by one or more sensors 58, 59 regarding information related to if water is flowing through the inline water module 56, if an ice making cycle is beginning or ending, and/or if water reservoir 70 has reached a maximum capacity of water. The system and method described herein are configured to maximize the benefit of any ozone produced. Thus, in some embodiments, ozone is infused into water reservoir 70 immediately prior to ice formation. In some embodiments, ozone is infused into water reservoir 70 when the water level has reached a maximum. In some embodiments, ozone is produced when a new ice making cycle is initiated. In some embodiments, ozone production halts when a new ice making cycle ends. FIG. 5 illustrates a front view of an assembly for an ice machine disinfecting apparatus 30, in accordance with one embodiment of the disclosure. In this example, water inlet 55 provides an inflow of fresh water into ozone venturi 44. Ozone generator 40 provides a supply of ozone via ozone infusion tube 60 into ozone venturi 44. Water continues to flow through pipe 48 past ultraviolet light source 42 for additional disinfection treatment. Subsequently, ozonated, disinfected water flows out of ozone generator housing 32 via water outlet 57 and may subsequently enter water reservoir 70 or flow directly into a compressor/condenser system for new ice formation. Ultraviolet light 42 may be on constantly or intermittently.

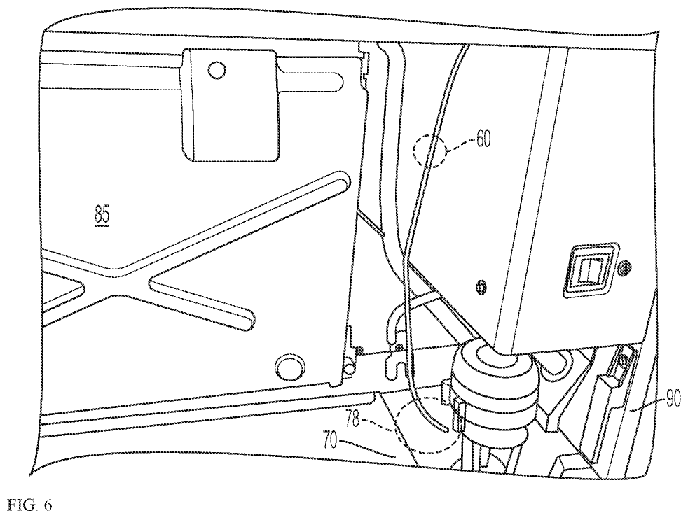

FIG. 6 illustrates a front perspective view of an interior portion of an ice machine cleaning apparatus 30, in accordance with an embodiment of the disclosure. Within ice maker 85, ozone flows into the water contained within water reservoir 70 via ozone infusion tube 60 and produces bubbles of ozone 78. Ice maker housing 90 contains these components.

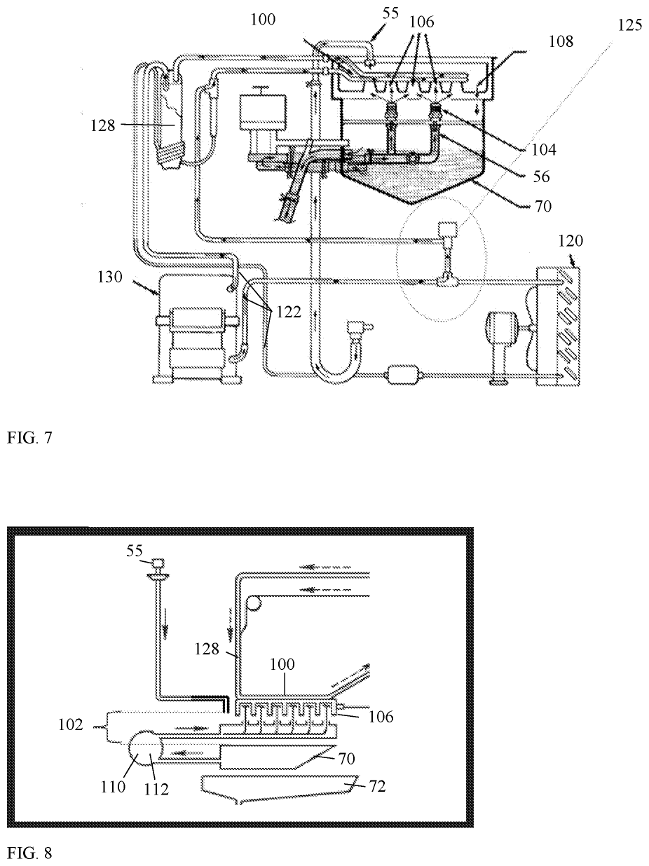

FIG. 7 illustrates an interior side view of an interior portion of ice machine cleaning apparatus 30, in accordance with an embodiment of the present disclosure. Within the ice maker, refrigerant fluid 128 cools evaporator 100 during new ice formation and then heats evaporator 100 to release the ice into an ice bin.

When ice maker 85 is turned on, water flows into the system via water inlet 55 and flows into water reservoir 70. Inline water module 56 pumps water from water reservoir 70 toward spray jets 104. Embodiments of the present disclosure can provide ozone into at least one of water inlet 55 and water reservoir 70. Spray jets 104 spray ozonated water toward ice cube molds 106. Compressor 130 increases the pressure of refrigerant 128 which raises its temperature. As refrigerant fluid 128 travels through an expansion valve (hot gas solenoid 125, in the illustrated embodiment) it begins to evaporate and turn into a gas. Refrigerant removes heat from the evaporator 100 causing ice to form. Ozonated water which is flowing over evaporator 100 begins to freeze in ice cube molds 106. Excess water that did not freeze in ice cube molds 106 can return to water reservoir 70 via weep hole 108. It can then be re-ozonated and returned to the evaporator.

As refrigerant 128 passes through narrow tubes 122, refrigerant 128 loses heat. After the ice cubes form, an evaporator sensor 101 (not shown) triggers a valve which tells compressor 130 to stop forcing heated refrigerant gas into condenser 120 and instead directs it to a bypass valve. From the bypass valve the hot gas cycles through evaporator 100 without cooling off and quickly heats up the evaporator and releases the ice from the cube molds 106 without melting it. The ice then falls into the ice bin where it can be scooped by hand or dispensed automatically. Once the ice is dropped, the process starts all over again.

FIG. 8 illustrates an exploded view of a portion of ice maker 85, in accordance with an embodiment of the present disclosure. Water inlet 55 provides water to evaporator 100. Heated refrigerant fluid 128 is configured to flow over evaporator 100 and encourage formation of water into ice cube molds 106. Water flows from water reservoir 70 into evaporator 100 via pump 110 which is configured with pump motor 112. An air gap 102 of at least 1.25″ is positioned between water reservoir 70 and evaporator 100. Excess water that does not form into ice can return to drain pan 72. In accordance with embodiments of the present disclosure, ozone may be infused into water inlet 55 or into water reservoir 70, or both.

Note that the processes in method 300 are shown in a particular order for ease of description. However, one or more of the processes may be performed in a different order or may not be performed at all (and thus be optional), in accordance with some embodiments. Numerous variations on method 300 and the techniques described herein will be apparent in light of this disclosure.

### FURTHER EXAMPLE EMBODIMENTS

The following examples pertain to further embodiments, from which numerous permutations and configurations will be apparent.

Example 1 is an ice maker comprising an ice making assembly including an evaporator mold configured for forming a plurality of ice pieces, a water reservoir, a water recirculator in fluid communication with the water reservoir and the evaporator mold, and an ozone generator configured and arranged to continuously infuse ozone into water in the water reservoir.

Example 2 includes the subject matter of Example 1, wherein the water recirculator comprises an inline water module.

Example 3 includes the subject matter of Example 2, wherein the water recirculator further comprises at least one of a pump and a weep hole.

Example 4 includes the subject matter of Example 2, wherein the inline water module is configured to provide water from the water reservoir to the evaporator mold.

Example 5 includes the subject matter of Example 4 and further includes spray jets positioned adjacent to a terminal end of the inline water module.

Example 6 includes the subject matter of Example 5, wherein the spray jets are configured to spray ozonated water toward the evaporator mold.

Example 7 includes the subject matter of Example 6, wherein the evaporator mold comprises a plurality of ice cube molds.

Example 8 includes the subject matter of Example 1 and further includes one or more sensors in electrical communication with a circuit board.

Example 9 includes the subject matter of Example 8, wherein the one or more sensors are configured to detect a call for new ice formation.

Example 10 includes the subject matter of Example 9, where the one or more sensors comprise one or more of (i) a flow valve sensor, (ii) a sensor configured to detect if water is flowing through a water inlet, (iii) a sensor configured to detect a beginning of new ice formation, or (iv) a sensor configured to detect an end of new ice formation.

Example 11 includes the subject matter of Example 1 and further includes a refresh switch configured to provide a timed production of ozone during period of infrequent ice formation.

Example 12 includes the subject matter of Example 11, wherein the refresh switch is a user override button configured external to the ice maker.

Example 13 includes the subject matter of Example 8, wherein the one or more sensors are configured to detect an ozone concentration.

Example 14 includes the subject matter of Example 8, wherein the one or more sensors are configured to detect at least one of (i) a temperature, (ii) a capacitance, (iii) a humidity, and (iv) movement.

Example 15 includes the subject matter of Examples 1-14 and further includes a shut-off switch on a cover, the shut-off switch configured to cease operation of the ozone generator.

Example 16 includes the subject matter of Examples 1-15, wherein the cover is made of an ozone-impervious material.

Example 17 is an apparatus comprising a cleaning apparatus comprising an ozone generator, at least one fluid line connecting an output from the ozone generator to at least one of a water inlet, a water recirculatory line, and a water reservoir, wherein the cleaning apparatus is configured for use with an ice machine.

Example 18 includes the subject matter of Example 17 and further includes a controller, wherein the controller is configured to drive ozone production during ice formation.

Example 19 includes the subject matter of Example 18 and further includes one or more sensors in electrical communication with a circuit board.

Example 20 includes the subject matter of Example 19, wherein the one or more sensors are configured to detect an ozone concentration.

Example 21 includes the subject matter of Example 17 and further includes a refresh switch configured to provide a timed production of ozone during period of infrequent ice formation.

Example 22 is a method of disinfecting an ice machine, the method comprising providing an ice machine configured for the production of ice, providing an ozone generator configured for producing ozone, directing an ozone delivery pathway from the ozone generator into at least one of a water supply and a water reservoir, and operating the ozone generator to deliver ozone to water inside the ice machine.

Example 23 includes the subject matter of Example 22, wherein the ice machine includes one or more sensors and the method further comprises detecting, by the one or more sensors, an ozone concentration, communicating a detected ozone concentration from the one or more sensors to a controller, and comparing, by the controller, the detected ozone concentration to a predetermined maximum value.

Example 24 includes the subject matter of Example 23, wherein the detected ozone concentration includes an ozone concentration from inside of the ice machine.

Example 25 includes the subject matter of Example 23 or Example 24, wherein the detected ozone concentration includes an ozone concentration outside of the ice machine.

Example 26 includes the subject matter of Examples 22-25, further comprising ceasing operation of the ozone generator if the detected ozone concentration exceeds the predetermined maximum value.

Example 27 includes the subject matter of Example 26, further comprising the controller communicating to a user a warning of an unsafe condition.

Example 28 includes the subject matter of Examples 23-27 and further includes detecting, by the one or more sensors, one or more condition of (i) a temperature, (ii) a capacitance, (iii) a humidity, and (iv) movement, communicating the one or more detected condition from the one or more sensors to the controller, and adjusting, by the controller, operation of the ozone generator.

Example 29 includes the subject matter od Example 28, wherein adjusting the operation of the ozone generator includes changing an operating level or operating time based at least in part on the temperature.

Example 30 includes the subject matter of Example 28 or Example 29, wherein adjusting the operation of the ozone generator includes changing an operating level or operating time of the ozone generator based at least in part on the humidity.

Example 31 includes the subject matter of Examples 28-30, wherein adjusting operation of the ozone generator includes ceasing operation of the ozone generator based the detected condition.

The foregoing description of example embodiments has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future-filed applications claiming priority to this application may claim the disclosed subject matter in a different manner and generally may include any set of one or more limitations as variously disclosed or otherwise demonstrated herein.

1. An ice maker comprising:
an ice making assembly including
an evaporator mold configured for forming a plurality of ice pieces;
a water reservoir;
a water recirculator in fluid communication with the water reservoir and the evaporator mold; and
an ozone generator in fluid communication with the water reservoir,
wherein the ozone generator is configured to provide a continuous supply of ozone during ice formation.

2. The ice maker of claim 1, wherein the water recirculator comprises an inline water module.

3. The ice maker of claim 2, wherein the water recirculator further comprises at least one of a pump and a weep hole.

4. The ice maker of claim 2, wherein the inline water module is configured to provide water from the water reservoir to the evaporator mold.

5. The ice maker of claim 4, further comprising spray jets positioned adjacent to a terminal end of the inline water module.

6. The ice maker of claim 5, wherein the spray jets are configured to spray ozonated water toward the evaporator mold.

7. The ice maker of claim 6, wherein the evaporator mold comprises a plurality of ice cube molds.

8. The ice maker of claim 1, further comprising one or more sensors in electrical communication with a circuit board.

9. The ice maker of claim 8, wherein the one or more sensors are configured to detect a call for new ice formation.

10. The ice maker of claim 9, where the one or more sensors comprise one or more of (i) a flow valve sensor, (ii) a sensor configured to detect if water is flowing through a water inlet, (iii) a sensor configured to detect a beginning of new ice formation, or (iv) a sensor configured to detect an end of new ice formation.

11. The ice maker of claim 1, further comprising a refresh switch configured to provide a timed production of ozone during period of infrequent ice formation.

12. The ice maker of claim 11, wherein the refresh switch is a user override button configured external to the ice maker.

13. The ice maker of claim 8, wherein the one or more sensors are configured to detect at least one of (i) a temperature, (ii) a capacitance, (iii) a humidity, (iv) movement, and (v) an ozone concentration.

14. A apparatus, comprising:
a cleaning apparatus comprising an ozone generator;
at least one fluid line connecting an output from the ozone generator to at least one of a water inlet, a water recirculatory line, and a water reservoir;
wherein the cleaning apparatus is configured for use with an ice machine, wherein the ozone generator is configured to provide a continuous supply of ozone during ice formation.

15. The apparatus of claim 14, further comprising a controller, wherein the controller is configured to drive ozone production during ice formation.

16. The apparatus of claim 14, further comprising a refresh switch configured to provide a timed production of ozone during period of infrequent ice formation.

17. A method of disinfecting an ice machine, the method comprising:
providing an ice machine configured for the production of ice;
providing an ozone generator configured for producing ozone;
directing an ozone delivery pathway from the ozone generator into at least one of a water supply and a water reservoir; and
operating the ozone generator to continuously deliver ozone to water inside the ice machine during ice production.

18. The method of claim 17, wherein the method further comprises:
detecting, by one or more sensors, an ozone concentration;
communicating a detected ozone concentration from the one or more sensors to a controller; and
comparing, by the controller, the detected ozone concentration to a predetermined maximum value.

19. The method of claim 18, further comprising:
detecting, by the one or more sensors, one or more condition of (i) a temperature, (ii) a capacitance, (iii) a humidity, and (iv) movement;
communicating the one or more detected condition from the one or more sensors to the controller; and
adjusting, by the controller, operation of the ozone generator.

20. The method of claim 19, wherein adjusting the operation of the ozone generator includes changing an operating level or operating time based at least in part on at least one of the temperature and the humidity.
